(19) United States
(12) Patent Application Publication
(10) Pub. No.: US 2022/0257160 A1
(43) Pub. Date: Aug. 18, 2022

(54) DEVICE FOR ARTERIAL PUNCTURE ASSISTANCE

(71) Applicant: University of Miami, Miami, FL (US)

(72) Inventors: RICARDO PALACIOS, Guayaquil (EC); ELIZABETH DECKLER, Miami, FL (US)

(73) Assignee: University of Miami, Miami, FL (US)

(21) Appl. No.: 17/627,370

(22) PCT Filed: Jul. 15, 2020
(86) PCT No.: PCT/US20/42181

§ 371 Date: Jan. 14, 2022

Related U.S. Application Data
(60) Provisional application No. 62/874,825, filed on Jul. 16, 2019.
(60) Provisional application No. 62/934,248, filed on Nov. 12, 2019.

(51) Int. Cl. A61B 5/15; A61B 5/153; A61B 5/00

(52) U.S. Cl. CPC A61B 5/150801; A61B 5/15003; A61B 5/6826; A61B 5/153

(57) ABSTRACT

Device for arterial puncture assistance. In an embodiment, the device comprises an upper component, lower component, and coupling mechanism that couples the lower component to the upper component. The upper component may comprise a platform configured to support a syringe. The lower component may comprise one or more finger holes, wherein each of the one or more finger holes is configured to receive a human finger therethrough, so as to enable contemporaneous stabilization of the lower component on the human finger and palpation of an arterial pulse by the human finger during an arterial puncture.

### CROSS-REFERENCE TO RELATED APPLICATIONS

This application claims priority to U.S. Provisional Patent App. No. 62/874,825, filed on Jul. 16, 2019, and U.S. Provisional Patent App. No. 62/934,248, filed on Nov. 12, 2019, which are both hereby incorporated herein by reference as if set forth in full.

### BACKGROUND

### Field of the Invention

The embodiments described herein are generally directed to arterial puncture, and, more particularly, to a device that aids in the insertion of a syringe into an artery.

### DESCRIPTION OF THE RELATED ART

Current methods of puncturing an artery with a syringe needle (e.g., for arterial blood sampling) can be prone to error. Depending on the skill level of the practitioner, these methods can take more time and needle insertions than desired.

For example, current methods of arterial blood sampling require the practitioner to palpate the radial artery pulse with one hand, while simultaneously, with the other hand, inserting a needle at a 45-degree angle into a segment of the radial artery that is distal to the site of palpation. This can be a difficult maneuver to achieve once—let alone, multiple times. Once the needle has punctured the desired artery, the practitioner must then hold the needle in a fixed position until the necessary volume of arterial blood has been extracted.

In addition, current methods may require the practitioner to insert the needle distal to the site of concurrent proximal palpation of the pulse. However, this increases the risk of needlestick injury to the practitioner's fingers, since the needle crosses over the practitioner's palpating fingers when it is inserted.

Emergency situations, in particular, can pose difficulties for practitioners in accurately and efficiently executing an arterial puncture. For example, complications from tense emergency situations can draw out the amount of time needed to sample arterial blood or place a continuous arterial blood gas (ABG) monitor prior to surgery. Thus, the collection of an arterial blood sample, placement of an ABG monitor, or any other necessary access to an artery can significantly delay the start of an emergency operation or other emergency procedure.

Thus, what is needed is an improved method for arterial puncture. In particular, one or more of the above problems could be alleviated by a device that is able to assist in consistently puncturing the radial artery at a chosen or optimum angle, enable movement of the needle in three-dimensional space, and/or aid the practitioner in searching for a radial artery while the needle is inserted into the skin of a patient, while maintaining stability of the needle at a chosen or optimum angle.

### SUMMARY

Accordingly, a device for assisting arterial punctures is disclosed. In an embodiment, the device comprises: an upper component comprising a platform configured to support a syringe; a lower component comprising one or more finger holes, wherein each of the one or more finger holes is configured to receive a human finger therethrough, so as to enable contemporaneous stabilization of the lower component on the human finger and palpation of an arterial pulse by the human finger during an arterial puncture; and a coupling component that couples the lower component to the upper component. The platform has a proximal end and a distal end defining a longitudinal axis, wherein the distal end is closer to a needle of the syringe when the syringe is supported by the platform.

The upper component may further comprise a finger guard that extends from the distal end of the platform. The finger guard may extend parallel to the platform. The finger guard may be movably attached to the distal end of the platform, such that the finger guard is capable of pivoting between a range of angles relative to the platform. The range of angles may comprise a 0-degree angle, in which the finger guard is parallel to the platform, and a 90-degree angle, in which the finger guard is perpendicular to the platform.

The platform may comprise an attachment component configured to releasably attach to a corresponding attachment component on the syringe and, when attached to the corresponding attachment component on the syringe, restrict movement of the syringe to a linear axis that is parallel to the longitudinal axis. The attachment component may comprise a T-shaped track extending from the proximal end to the distal end of the platform.

A cross section of the platform, in a plane that is perpendicular to the longitudinal axis, may be semi-circular. The platform may be tapered, such that a cross section of the platform in a plane that is perpendicular to the longitudinal axis consists of a smaller portion of a circle at the proximal end than at the distal end. A cross section of the platform, in a plane that is perpendicular to the longitudinal axis, may be circular from the proximal end to the distal end, wherein a diameter of the cross section at the proximal end is not equal to a diameter of the cross section at the distal end.

The lower component may comprise at least two finger holes. The lower component may consist of two finger holes. A first one of the at least two finger holes may have a different inner diameter than a second one of the at least two finger holes. The first finger hole may be configured to receive a human middle finger, and the second finger hole may be configured to receive a human index finger. Each of the at least two finger holes may be configured to only surround a proximal phalanx of a human finger. Each of the at least two finger holes may have a proximal end and a distal end, wherein the distal end is closer to a needle of the syringe when the syringe is supported by the platform, and wherein the distal end of each of the at least two finger holes is tapered. The distal end of each of the at least two finger holes may be tapered, such that a length of each finger hole decreases from a top to a bottom of the finger hole. Each of the at least two finger holes may comprise an annular grip within the finger hole, wherein each annular grip comprises a tube of compressible material configured to receive a human finger therethrough. Alternatively, the lower component may consist of a single finger hole configured to receive a human finger therethrough.

The coupling component may be configured to enable movement of the upper component relative to the lower component, when manual force is applied, such that the upper component is movable through a range of angles relative to the lower component, wherein the coupling component is configured to prevent movement of the upper component relative to the lower component when no manual force is applied. The coupling component may comprise a ball and socket, such that the upper component is movable through a range of angles in three dimensions relative to the lower component.

In an embodiment, the device comprises: an upper component comprising a platform configured to support a syringe, wherein the platform has a proximal end and a distal end defining a longitudinal axis, wherein the distal end is closer to a needle of the syringe when the syringe is supported by the platform, and wherein the upper component further comprises a finger guard that extends from the distal end of the platform; a lower component comprising one or more finger holes, wherein each of the one or more finger holes is configured to receive a human finger therethrough, so as to enable contemporaneous stabilization of the lower component on the human finger and palpation of an arterial pulse by the human finger during an arterial puncture; and a coupling component that couples the lower component to the upper component, and wherein the coupling component comprises a ball and socket, such that the upper component is movable through a range of angles in three dimensions relative to the lower component.

### BRIEF DESCRIPTION OF THE DRAWINGS

The details of the present invention, both as to its structure and operation, may be gleaned in part by study of the accompanying drawings, in which like reference numerals refer to like parts, and in which:

FIG. 1 illustrates a perspective view of a device being used to assist an arterial puncture, according to an embodiment;

FIGS. 2A-2F illustrate various views of a device for assisting arterial punctures, according to an embodiment; and

FIG. 3-6 illustrate various features of a device for assisting arterial punctures, according to embodiments.

### DETAILED DESCRIPTION

In an embodiment, a device for improving arterial punctures (e.g., for arterial blood sampling) is disclosed. After reading this description, it will become apparent to one skilled in the art how to implement the invention in various alternative embodiments and alternative applications. However, although various embodiments of the present invention will be described herein, it is understood that these embodiments are presented by way of example and illustration only, and not limitation. As such, this detailed description of various embodiments should not be construed to limit the scope or breadth of the present invention as set forth in the appended claims.

The disclosed device can assist a medical practitioner or other user (collectively referred to herein as a “practitioner”) in accurately and efficiently puncturing a radial artery with a needle of a syringe or other medical instrument, while simultaneously palpating the pulsation of a radial artery with his or her fingers at a position that is distal to the insertion site of the needle (e.g., in front of the insertion site). In an embodiment, the device has a lower component that comprises a finger holder that provides the practitioner with stability and safety during use of the device. The finger holder may have at least two finger holes configured to receive two or more of the practitioner's fingers. For example, the finger holder may comprise or consist of two finger holes, configured to receive a pair of adjacent fingers, such as the practitioner's middle and index fingers. Each finger hole may have a size or diameter that is based on the average diameter of the respective finger for which it was designed. In an embodiment, each finger hole could comprise a sponge or sponge-like material within the finger hole to enhance the fit of the finger hole around the practitioner's finger. The practitioner may insert his or her fingers into the finger holders of the lower component, and hold the finger holder firmly against the wrist of a patient.

The device may also have an upper component with a platform used to guide the syringe with the needle towards the artery. The syringe platform may have a semi-circular or circular cross section, extending from a proximal end to a distal end, that is sized or otherwise configured to receive and support a syringe. The syringe platform may also have a finger guard affixed to one end. The finger guard may be adjustable. In an embodiment, the platform may comprise an attachment mechanism (e.g., a male/female T-track structure) that enables joinder of the syringe to the platform so as to limit movement of the syringe to a linear axis that is parallel to the longitudinal axis of the platform.

The lower and upper components can be coupled or joined at an interface by a coupling mechanism, such as a joint (e.g., a ball-and-socket mechanism), magnetic forces, and/or the like. The friction at the interface may be set so that it is high enough to maintain a desired angle between the lower and upper components (e.g., between the finger holes and syringe platform), but low enough that the upper component can be moved relative to the lower component in response to a minimal manual force applied by the practitioner. While the coupling mechanism preferably enables relative movement between the upper and lower components, in an alternative embodiment, the coupling mechanism may comprise a fixed structure that does not enable relative movement between the upper and lower components. In any case, upper component provides the practitioner with a platform on which the syringe can be seated and stabilized at a desired angle with respect to the patient's artery, thereby reducing or eliminating undesired movement of the syringe. Thus, the device can improve the consistency in angle and stability of the syringe during arterial blood sampling or any other procedure involving insertion of a needle (e.g., for insertion or extraction of fluids and/or other substances).

Advantageously, the disclosed device may reduce unwanted movements, decrease the risk of the needle missing the desired puncture site on the patient, and/or decrease the risk of injuring structures around an artery (e.g., a radial nerve). In addition, by keeping the needle still during extraction, the size of the puncture in the radial artery can be reduced, since less movement will be present to stretch the puncture. Furthermore, the coupling mechanism between the upper and lower components allows the practitioner to selectively move the syringe in any direction while inserted and while still maintaining a certain angle. This enables the practitioner to search for an artery, if needed, without having to pull out and reinsert the needle into the skin.

### 1. Example Embodiment

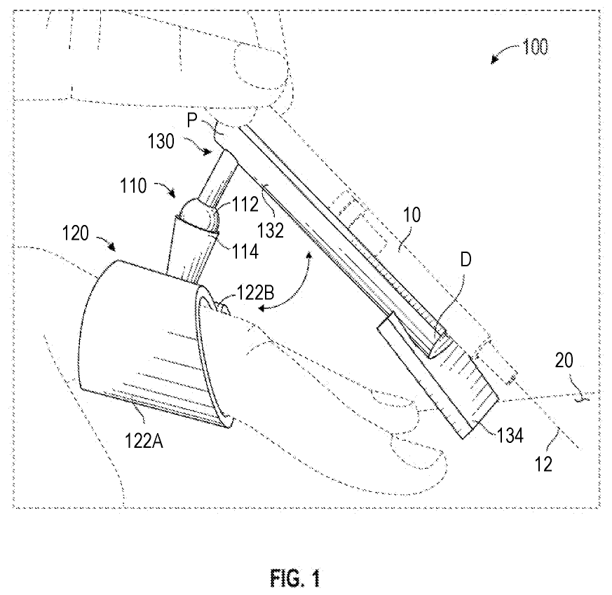

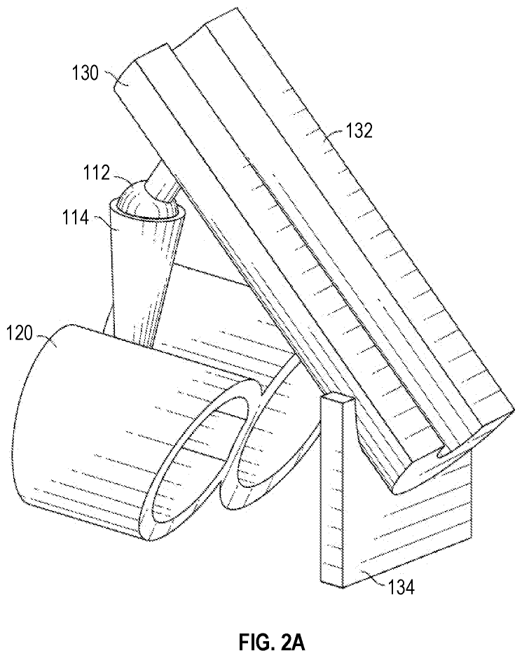

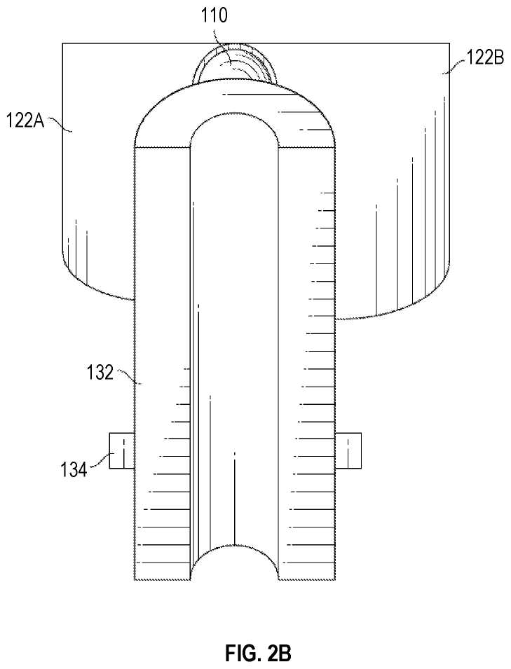

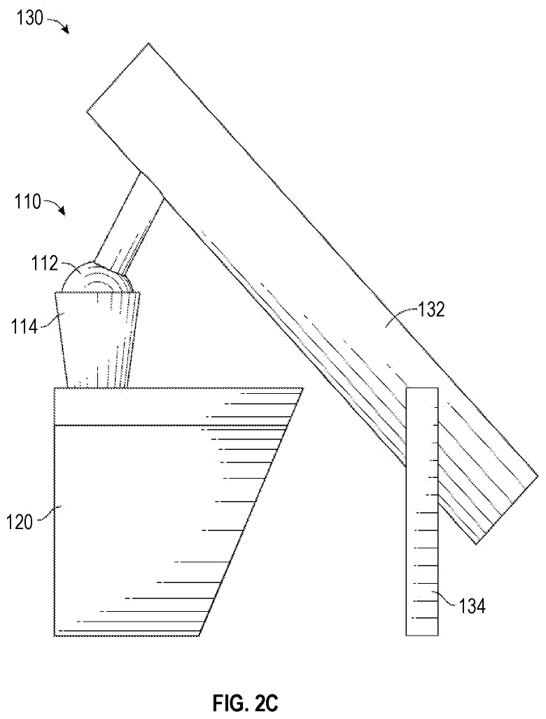

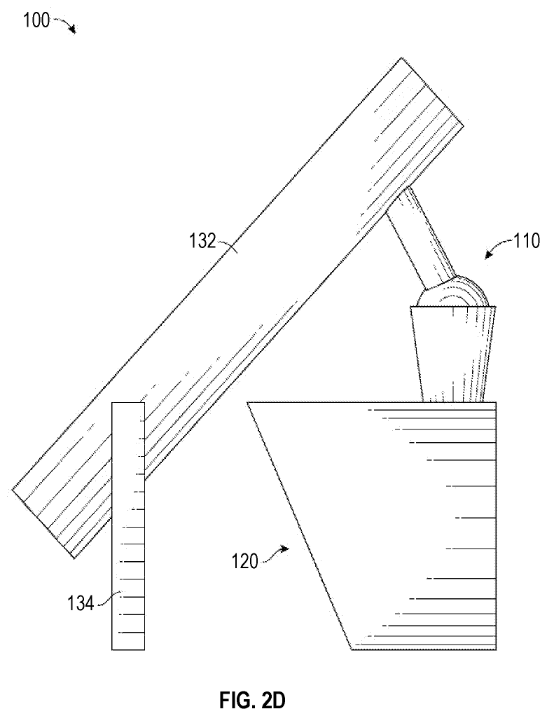

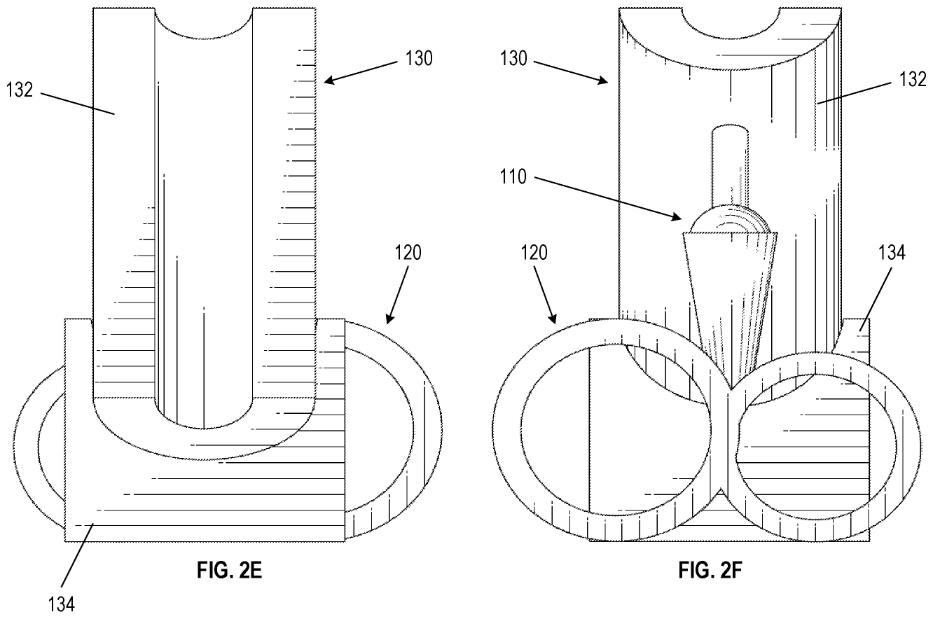

FIG. 1 illustrates a perspective view of a device 100 being used to assist an arterial puncture, according to an embodiment. In addition, FIG. 2A illustrates a top perspective view of device 100, FIG. 2B illustrates a top plan view of device 100, FIGS. 2C and 2D illustrate opposite side views of device 100, FIG. 2E illustrates a front view of device 100 (i.e., facing a distal end), and FIG. 2F illustrates a rear view of device 100 (i.e., facing a proximal end), according to an embodiment.

In FIG. 1, device 100 is illustrated on a practitioner's hand and with an external syringe 10 having a needle 12 for insertion into a patient's skin 20. Advantageously, device 100 stabilizes syringe 10, for example, during an arterial puncture for an arterial blood draw. Device 100 comprises a lower component 120 and an upper component 130, coupled by a coupling component 110. Lower component 120 enables the practitioner to stably and safely hold syringe 10 as it is supported by upper component 130. In practice, a practitioner may rest lower component 120 on the body of the patient (e.g., the patient's forearm), during the procedure, in order to stabilize device 100.

In the illustrated embodiment, lower component 120 comprises a finger holder with two finger holes 122A and 122B. As illustrated, finger holes 122 may be circular in cross section to accommodate the typical human finger. However, finger holes 122 could have a different cross-sectional shape (e.g., oval, triangle, square, rectangle, pentagon, hexagon, heptagon, octagon, or any other multi-sided polygon). In addition, in an alternative embodiment, the finger holder could be replaced with any other structure that assists a practitioner in anchoring device 100 to a patient's body.

As an example, finger hole 122A may be designed for a practitioner's middle finger, and finger hole 122B may be designed for a practitioner's index finger. The diameter of each finger hole 122 may be designed based on the average diameter for the particular finger that it was designed to receive, such that different finger holes 122 may have different diameters. For example, finger hole 122A, which is designed to slide over the practitioner's middle finger, may have a smaller diameter than finger hole 122B, which is designed to slide over the practitioner's index finger. In this case, device 100 may be manufactured in both right-handed and left-handed embodiments. Alternatively, all finger holes 122 may have the same diameter based on the average diameter of the fingers that they were designed to receive. In this case, the same device 100 may be used by both right-handed and left-hand practitioners. In either case, device 100 may be manufactured in different sizes, such as a small size (e.g., with finger holes 122 having a diameter that is smaller than the average diameter of human fingers), a medium size (e.g., with finger holes 122 having a diameter that is equal to the average diameter of human fingers), and a large size (e.g., with finger holes 122 having a diameter that is larger than the average diameter of human fingers).

While the illustrated embodiment consists of only two finger holes 122, it should be understood that alternative embodiments may comprise one, three, four, or five finger holes 122. However, device 100 preferably has at least two finger holes 122 to provide more stability over an embodiment with only a single finger hole 122, which could allow inadvertent rotation of the finger holder around the practitioner's finger. In addition, an embodiment that consists of only two finger holes 122 may be preferable, since it may fit a wider range of practitioners' hand sizes than embodiments with three or more finger holes 122. Thus, two finger holes 122 appropriately balances stability with flexibility.

Furthermore, while finger holes 122 are illustrated as receiving the middle finger (e.g., third finger) and index finger (e.g., second finger) of a practitioner, finger holes 122 may be configured to receive different fingers. For example, finger hole 122A may be designed to receive the fourth finger (e.g., the ring finger) while finger hole 122B may be designed to receive the middle finger. However, the middle and index fingers may provide the greatest stability and control for the majority of practitioners.

The length of finger holes 122 may be designed so that a substantial portion of the practitioner's finger extends out of the distal end of each finger hole 122. For example, the length of each finger hole 122 may be less than or equal to the length of the average person's proximal phalanx (i.e., from the first knuckle, joining the hand to the finger, to the second knuckle) on the respective finger. This enables the practitioner to bend his or her finger at the second and third knuckles, so that, for example, the practitioner can feel the patient's pulse using the same fingers that are extending through finger holes 122. Each finger hole 122 can have the same length or have different lengths, for example, corresponding to the average length of the proximal phalanx of the finger for which the finger hole 122 is designed, the average position of the second knuckles of the fingers relative to each other, or to provide one finger with more mobility than another finger (e.g., with shorter lengths representing more mobility, and greater lengths representing less mobility). Thus, finger hole 122A (e.g., for the middle finger) may be shorter than finger hole 122B (e.g., for the index finger), finger hole 122A may be longer than finger hole 122B, or finger hole 122A may be substantially the same length as finger hole 122B.

In an embodiment, one or more of finger holes 122 may be tapered on at least one end and/or non-tapered on at least one end. For example, in the illustrated embodiments, finger holes 122 are both non-tapered on the proximal ends (i.e., closer to the first knuckle) and tapered on their distal ends (e.g., farther from the first knuckle). Alternatively, each finger hole 122 could be tapered on both the proximal end and distal end, non-tapered on both the proximal end and distal end, or tapered on the proximal end and non-tapered on the distal end. Furthermore, the proximal and/or distal ends of different finger holes 122 could be tapered and/or non-tapered differently from other finger holes 122. The tapering may be in any direction. For example, in the embodiment illustrated in FIG. 1, finger holes 122 have a greater length at the bottom of finger holes 122 (e.g., farther from upper component 130) and taper to a smaller length at the top of finger holes 122 (e.g., closer to upper component 130). Alternatively, as illustrated in FIGS. 2A-2F, the tapering could comprise a smaller length at the bottom of finger holes 122 that tapers to a greater length at the top of finger holes 122. The direction of tapering in FIGS. 2A-2F may be preferable over the direction of tapering in FIG. 1, because it better enables the practitioner's fingers to bend downward at the knuckles (e.g., second and third knuckles), for example, to take a patient's pulse, while simultaneously covering and thereby protecting the practitioner's knuckles from needlestick injuries by needle 12.

Upper component 130 of device 100 may comprise a platform 132 that is configured to guide a syringe 10 during a procedure. Specifically, syringe 10 rests stably on platform 132 and can slide towards a distal end D to puncture a patient's skin 20 with needle 12 at a desired angle and insertion site. In the illustrated embodiment, platform 132 is formed as a portion of a tube or cylinder having a substantially semi-circular or U-shaped cross section, in a plane that is orthogonal to the longitudinal axis of platform 132, extending from proximal end P (e.g., closer to the practitioner and farther from needle 12 during use) to distal end D (e.g., farther from the practitioner and closer to needle 12 during use). Alternatively, platform 132 could be a tube or cylinder having a substantially circular cross section, with the disadvantage that platform 132 may not be able to accommodate as many different sizes of syringes 10 as an embodiment with a substantially semi-circular cross section. In such an embodiment, the diameter of the circular cross section at one end of platform 132 may be the same or different than the diameter of the circular cross section at the opposite end of platform 132. As yet another alternative, the cross section of platform 132 may have a shape that is not semi-circular or circular, such as a triangle, square, rectangle, pentagon, hexagon, heptagon, octagon, or any other multi-sided polygon, or a portion (e.g., half) of any such shapes. As used herein, “semi-circular” or “circular” may also refer to a semi-ovular or ovular shape. In addition, “semi-circular” should be understood to include a cross section that is an arc forming half of a circle or oval, less than half of a circle or oval, or slightly more than half of a circle or oval.

The length of platform 132 (i.e., the distance from the edge of proximal end P to the edge of distal end D) may be based on the length of the syringe(s) 10 which platform 132 is intended to support. In other words, the length of platform 132 may be chosen to stably support a single size of syringe 10 or a plurality of different sizes of syringes 10, so as to prevent syringe 10 from wobbling or tipping off the proximal end P or distal end D of platform 132.

Similarly, the radius, width, and/or depth of the cross section of platform 132 may be chosen to stably support a single diameter of syringe or a plurality of different diameters of syringes 10 (e.g., to prevent syringe 10 from rolling laterally within or off of platform 132). For example, the inner radius of the semi-circular cross section may be greater than or equal to the outer radius of a syringe 10 to be used and wide enough to encompass the full diameter or a substantial portion of the diameter of the syringe 10 to be used.

However, it should be understood that platform 132 may be implemented with any length or cross-sectional shape that is suitable to for the syringe 10 or other medical instrument to be supported on platform 132. For example, the length of platform 132 may be based on the length of the syringe 10 to be used, and the cross section of platform 132 may substantially match at least a portion of the cross section of the syringe 10 to be used. In this manner, platform 132 may be designed to accommodate any variety of different shapes and sizes of syringes 10 or other medical instruments.

In an embodiment, the top surface of platform 132 that interfaces with and contacts syringe 10 may be textured or made of or coated with a material or substance designed to increase friction between the surface and syringe 10. For example, the surface may be made of or coated with a material or substance that has a stickiness configured to lightly, releasably, and temporarily hold syringe 10. The texturing or coating may be provided across the whole top surface of platform 132 or just one or more partial regions of the top surface of platform 132. The increase in friction can improve the stability of syringe 10, which can be especially helpful when the practitioner is performing a procedure under significant stress (e.g., with trembling hands), such as during an emergency.

Upper component 130 of device 100 may also comprise a finger guard 134 that extends downward at an angle from platform 132 at or near distal end D. Finger guard 134 protects the practitioner's fingers from injury from inadvertent needlesticks from needle 12. For example, finger guard 134 separates a region in which the distal ends of practitioner's fingers are present (e.g., extending from finger holes 122) and a region in which needle 12 is present, in order to prevent contact between the practitioner's fingers and needle 12. Finger guard 134 should be configured to protect the practitioner's fingers at all orientations and relative angles of platform 134. To this end, finger guard 134 may be varied in size, shape, width, and/or length, for a given application, to maximize protection of the practitioner's fingers. Furthermore, finger guard 134 may extend from platform 132 at any suitable angle (e.g., in a range of greater than 0 degrees and less than 90 degrees), depending on the intended application. For example, finger guard 134 may extend from platform 132 at an approximate 45-degree angle. As another example, finger guard 134 may extend from platform 132 at an approximate 90-degree angle. As yet another example, finger guard 134 may extend parallel with platform 132 and needle 12 at a 0-degree angle, to allow the practitioner's fingers to safely rest in close proximity to needle 12 (i.e., without touching or coming into the path of needle 12).

In an embodiment, finger guard 134 may be pivotally connected to platform 132, so that the angle between finger guard 134 and platform 132 may be manually adjusted through a range of varying degrees. For example, finger guard 134 may be adjustable from a range of 0 degrees (e.g., substantially parallel to the bottom surface of platform 132) to 90 degrees (e.g., substantially perpendicular to the bottom surface of platform 132). Thus, a practitioner may adjust finger guard 134 to any inclination that corresponds to the practitioner's preferred angle.

Additionally or alternatively, finger guard 134 may be attachable and detachable from platform 132. In such an embodiment, a practitioner could detach finger guard 134 from platform 132 if desired (e.g., for improved maneuvering) or attach finger guard 134 to platform 132 if desired (e.g., for improved safety). In addition, the practitioner could switch a plurality of finger guards 134 in or out, as needed or desired. For example, a practitioner with larger fingers could replace a smaller finger guard 134 with a larger finger guard 134 for added protection, or could replace a larger finger guard 134 with a smaller finger guard 134 for additional maneuverability.

In an embodiment, lower component 120 and upper component 130 are coupled together via coupling mechanism 110. In the embodiment illustrated in FIGS. 1-2F, coupling mechanism 110 comprises a ball-and-socket mechanism. Specifically, a ball-and-rod portion 112 is movably joined to a socket portion 114 by inserting the ball at the end of the rod of ball-and-rod-portion 112 into a socket of socket portion 114. The socket of socket portion 114 allows the ball of ball-and-rod portion 112 to rotate freely, through a substantially hemispherical range of positions, in three-dimensions within the socket. In an embodiment, socket portion 114 may allow rotation of ball-and-rod portion 112, such that upper component 130 may be rotated in 360 degrees in a horizontal plane. Alternatively, coupling mechanism 110 could be designed to restrict rotation of upper component 130 to a range that is less than 360 degrees in the horizontal plane, for example, to prevent syringe needle 12 from facing the practitioner. Similarly, socket portion 114 may also enable rotation of ball-and-rod portion 112, such that upper component 130 may be rotated in 180 degrees in a vertical plane, or restrict rotation of upper component 130 to a range that is less than 180 degrees in the vertical plane.

While ball-and-rod portion 112 extends from upper component 130 and socket portion 114 extends from lower component 130 in the illustrated embodiment, these portions may be reversed in an alternative embodiment, such that ball-and-rod portion 112 extends from lower component 120 and socket portion 114 extends from upper component 130. Thus, no specific distinction is made as to which component corresponds to the male portion of the ball-and-socket mechanism and which component corresponds to the female portion of the ball-and-socket mechanism.

The socket of socket portion 114 may receive the ball of ball-and-rod portion 112 in an interference fit. Thus, the ball-and-socket mechanism exhibits a degree of friction between socket portion 114 and ball-and-rod portion 112. In an embodiment, this degree of friction is sufficiently high to maintain a desired angle of platform 132, with minimal to no movement of platform 132, while syringe 10 is being supported by platform 132. In other words, the degree of friction should be such that, even when syringe 10 is being held on platform 132, platform 132 does not move, relative to any other component of device 100, unless the practitioner intentionally applies a manual force to platform 132. Of course, platform 132 may still move along with device 100, for example, as the practitioner moves his or her hand or fingers. It should be understood that, as used herein, the “angle” of platform 132 may refer to the angle of the longitudinal axis of platform 132 relative to lower component 120, the practitioner's fingers, the patient, or the insertion site of needle 52 into the patient's skin 20.

In an alternative embodiment, coupling mechanism 110 may be implemented with a magnet. For example, instead of or in addition to a ball-and-socket mechanism, one or both of portion 112 on upper component 130 and portion 114 on lower component 120 may comprise a magnet that attracts a magnet or metallic surface of the other portion to hold the adjacent portions together, while still permitting mobility between the two portions 112 and 114. The strength of the magnet(s) may be selected as appropriate to achieve the desired mobility between portions 112 and 114. In further alternative embodiments, coupling mechanism 110 may comprise a twist-lock coupling, bolt-nut coupling, bendable coupling (e.g., rubber-coated wire), and/or the like. As yet another alternative, lower component 120 and upper component 130 may be one continuous structure, with platform 132 of upper component 130 having a fixed angle relative to lower component 120.

### 2. Alternative Features

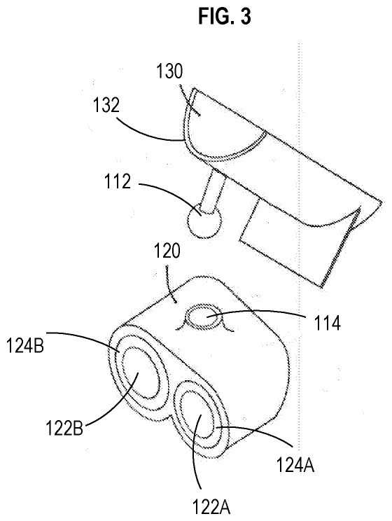

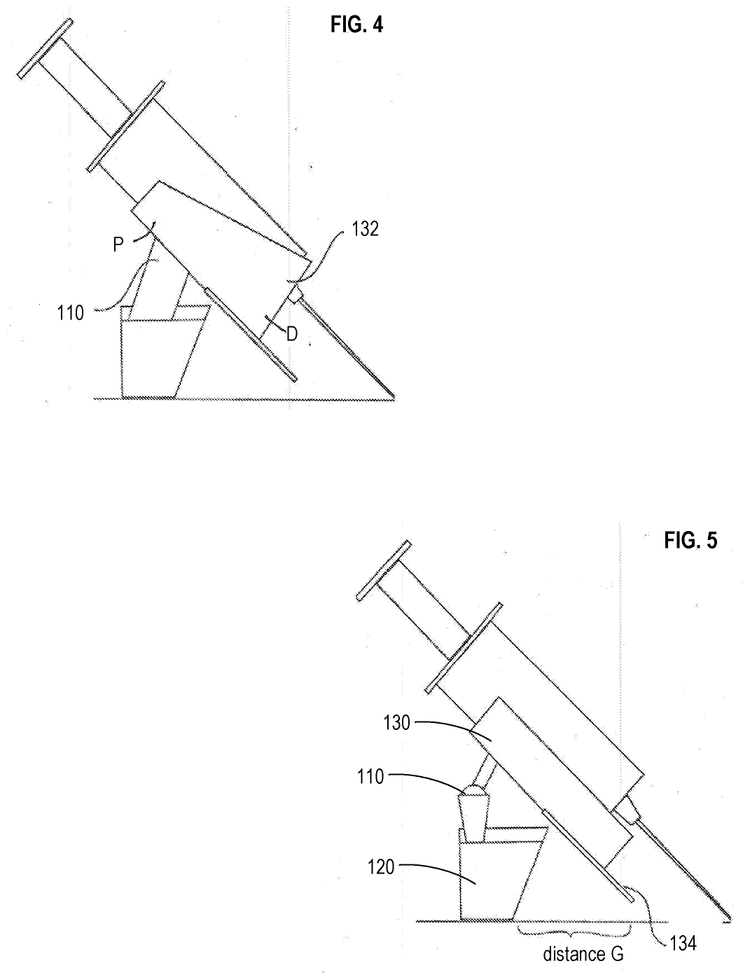

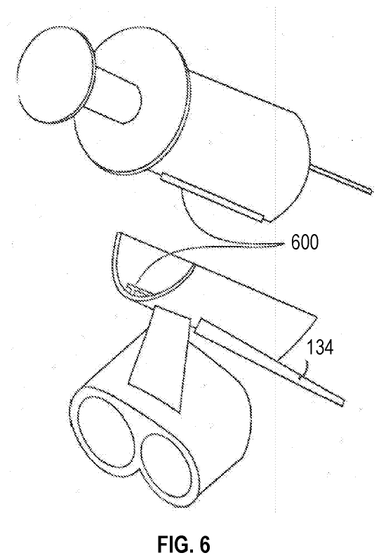

FIGS. 3-6 illustrate various alternative features of device 100. Specifically, FIG. 3 illustrates a perspective view of the de-coupled components of device 100, FIGS. 4 and 5 illustrate side views of device 100, and FIG. 6 illustrates a rear perspective view of device 100, according to various alternative embodiments. It should be understood that not all embodiments are described herein, and that embodiments may comprise any combination of any of the features described with respect to any of the embodiments described herein. For example, the fact that a first feature may be described with respect to an embodiment that comprises a second feature does not mean that all embodiments with the first feature must also comprise the second feature, or vice versa. Rather the first feature may be combined, with or without the second feature, with any other feature in any other embodiment to create an embodiment that is not explicitly described herein. In addition, the first feature may be omitted entirely from an embodiment or used on its own (i.e., without any other described features) in an embodiment.

In the embodiment illustrated in FIG. 3, lower component 120 comprises an annular grip 124 within each finger hole 122. Each annular grip 124 is tubular or substantially cylindrical with an outer diameter that corresponds to the inner diameter of its respective finger hole 122. Annular grip 124 may be made from a soft or compressible (e.g., sponge or sponge-like) material, such as rubber or similar material. The inner diameter of each annular grip 124 may be sized according to the average diameter of the finger which the annular grip 124 is intended to receive. For example, the inner diameter of each annular grip 124 may be slightly smaller than the average diameter of the finger which the annular grip 124 is intended to receive. Thus, as a practitioner inserts his or her finger into a given finger hole 122, the material of annular grip 124 in that finger hole 122 may compress outwards, such that when the practitioner's finger is received within finger hole 122, annular grip 124 surrounds the finger to form a tight, friction fit around the finger. In this manner, annular grips 124 enable finger holes 122 to securely accommodate a wide range of different finger diameters. Annular grips 124 may be fixed within their respective finger holes 122 via adhesive and/or another fixation mechanism. It should be understood that the inner and/or outer diameters of each annular grip 124 may be different than the inner and/or outer diameters of the other annular grip(s) 124, because each annular grip 124 may be sized to fit a different finger and/or fit within a differently sized finger hole 122. For example, annular grip 124A may have smaller inner and outer diameters than annular grip 124B, larger inner and outer diameters than annular grip 124B, a smaller inner diameter but larger outer diameter than annular grip 124B, or a larger inner diameter but smaller outer diameter than annular grip 124B.

Notably, platform 132 of the embodiment illustrated in FIG. 3 also has a thinner cross section and greater inner radius than the embodiments illustrated in FIGS. 1-2F. Accordingly, device 100 in FIG. 3 may support a syringe 10 with a larger diameter than device 100 in FIGS. 1-2F. It should be understood that the inner and/or outer radiuses of different embodiments of platform 132 in device 100 may be selected to fall within any practical range according to the size of syringe 10 or other medical instrument that device 100 is intended to support. Generally, a platform 132 with an inner radius that closely matches the outer radius of the intended syringe 10 will provide greater stability and control during insertion of needle 12. The size and shape of platform 132 may also be dictated by the procedure to be performed. For example, different procedures may require greater levels of stability and/or control.

FIG. 3 also illustrates a slightly different socket portion 114 for coupling mechanism 110 than the socket portion 114 illustrated in FIGS. 1-2F. Specifically, socket portion 114 in the embodiment illustrated in FIG. 3 has a lower profile, but is still shaped and sized to stabilize upper component 130 while enabling flexible control of the relative angle of upper component 130. It should be understood that many other variations of the ball-and-socket mechanism of coupling mechanism 110 and many other variations of coupling mechanism 110 itself are possible, as long as coupling mechanism 110 is structurally capable of stably supporting upper component 130. It should also be understood that many variations of lower component 120, including omission of lower component 120 entirely, are possible. However, it is preferable to have a lower component 120 that can at least rest against a patient's body (e.g., forearm) to help stabilize platform 132 of upper component 130, while enabling the practitioner to complete the relevant procedure (e.g., any procedure using syringe 10) safely and effectively.

In the embodiment illustrated in FIG. 4, coupling mechanism 110 is a structure that fixes the orientation and angle of upper component 130 relative to lower component 120. Thus, unlike the previously described embodiments, platform 132 of upper component 130 cannot be rotated with respect to lower component 120. However, in this embodiment, coupling mechanism 110 may still allow platform 132 to slide along the longitudinal axis defined by distal end D and proximal end P. Thus, a practitioner may slide platform 132 towards a patient (i.e., in the direction of distal end D) or away from the patient (i.e., in the direction of proximal end P). The length of coupling mechanism 110, defining the distance between lower component 120 and upper component 130, may be set to achieve any desired angle for syringe 10. For example, a coupling mechanism 110 with a shorter length will generally achieve a more obtuse angle (e.g., flatter approach with respect to the insertion site), whereas a coupling mechanism 110 with a greater length will generally achieve a more acute angle (e.g., higher angle with respect to the insertion site).

In addition, platform 132 in the embodiment illustrated in FIG. 4 has a cross section that varies from distal end D to proximal end P. Specifically, platform 132 has a deeper cross section at its distal end D than at its proximal end P. For example, at distal end D, the cross section of platform 132 may be almost circular (e.g., crescent shaped) or substantially circular, whereas at the proximal end P, the cross section of platform 132 may be semi-circular. Consequently, as illustrated in FIG. 4, the sides of platform 132 are tapered from a shallower cross section at proximal end P to a deeper cross section a distal end D. This configuration enables more flexibility in the movement of syringe 10 as syringe 10 is inserted into platform 132 (i.e., due to the shallowness of platform 132 at proximal end P), while fully limiting syringe 10 once syringe 10 has been completely inserted into platform 132 (i.e., due to the depth of platform 132 at distal end D).

In the embodiment illustrated in FIG. 5, finger guard 134 is substantially parallel to the longitudinal axis of platform 132. Notably, there is a distance G between the distal end of lower component 120 and the distal end of finger guard 134, which may be increased or decreased by adjusting upper component 130 relative to lower component 120 via coupling mechanism 110. The range of distance G may be set so as to comfortably accommodate the average finger size while allowing the fingers within finger holes 122 of lower component 120 to perform actions, such as taking the patient's pulse, during a procedure, such as arterial blood sampling.

In the embodiment illustrated in FIG. 6, platform 132 comprises an attachment mechanism 600. As shown, attachment mechanism 600 may comprise a T-track structure with a T-shaped track or rail, referred to herein as the “male” portion, and a “female” portion that receives and holds onto the T-shaped track, so as to slide along the T-shaped track. In FIG. 6, the male portion is integrated into platform 132, and the female portion is attached to syringe 10. Thus, a practitioner may slide the female portion of syringe 10 onto the male portion (e.g., T-shaped track) of platform 132. In an alternative embodiment, the female portion may be integrated into platform 132, and the male portion may be attached to syringe 10. In other alternative embodiments, a different attachment mechanism 600 may be used to releasably attach syringe 10 to platform 132, including track-based mechanisms and non-track-based mechanisms.

In any case, attachment mechanism 600 allows the syringe 10 to be attached and detached from platform 132. Attachment mechanism 600 is configured to, when syringe 10 is attached to platform 132, restrict the movement of syringe 10 to a linear movement along or parallel to the longitudinal axis of platform 132. In other words, attachment mechanism 600 guides syringe 10 along a linear path on platform 132 in either the proximal or distal direction. For example, attachment mechanism 600 locks or secures the downward path of syringe 10 during insertion of syringe 10 into the patient's skin 20. This restriction of movement can provide greater stability, control, and safety. After a procedure, the practitioner may remove syringe 10 by decoupling the components of attachment mechanism 600 (e.g., sliding the female portion off of the track or other male portion).

In an embodiment (not shown), finger hole(s) 122 may be sized, shaped, and/or otherwise configured to accommodate multiple fingers. For example, instead of a plurality of finger holes, each configured to receive a single finger, lower component 120 may comprise one or more finger holes that are each configured to receive two or more fingers. For example, lower component could consist of a single finger hole 122 that is sized and shaped to receive an average pair of middle and index fingers. In such an embodiment, the finger hole 122, which is configured to receive a plurality of fingers, may comprise semi-circular partitions to separate the individual fingers.

In an embodiment (not shown), device 100 may comprise a lower component 120 that has no holes or no holes configured to receive a finger. In this case, lower component 120 can comprise any apparatus that helps a practitioner to feel and/or locate a patient's pulse. As another alternative, lower component 120 may be omitted from device 100 altogether. In this case, device 100 may comprise or consist of upper component 130 and use anatomic landmarks to determine the insertion site for needle 12.

### 3. Example Usage

In a preferred embodiment, device 100 enables platform 132—and therefore, a syringe 10 supported on platform 132—to be moved, relative to lower component 120, through multiple degrees of freedom and angles to facilitate a medical procedure. The practitioner manually moves platform 132, within his or her discretion, to set it to a desired angle for a procedure (e.g., an arterial blood draw) relative to the patient's skin. The practitioner may do this before or after positioning syringe 10 on platform 132 (e.g., placing or sliding syringe 10 onto platform 132, attaching syringe 10 to platform 132 using attachment mechanism 600, etc.). In some cases, the practitioner may utilize a protractor or other device to precisely set the appropriate angle, prior to use.

Before or after setting the relative angle of platform 132, the practitioner may insert his or her fingers into respective finger holes 122. For example, in a preferred embodiment, device 100 may comprise a finger hole 122A for a middle finger and a finger hole 122B for an index finger. Thus, the practitioner may insert his or her middle finger into finger hole 122A and index finger in finger hole 122B. In embodiments which comprise annular grips 124, annular grips 124 snugly hold the practitioner's fingers within finger holes 122.

With his or her fingers through finger holes 122, thereby stably holding device 100, the practitioner may take the patient's pulse using those same fingers. For example, using the portions (e.g., distal phalanx and/or middle phalanx) of the practitioner's middle and index fingers extending out of the distal end of finger holes 122, under platform 132 (e.g., within the space defined by distance G in FIG. 5), the practitioner may apply pressure to the patient's artery so as to identify the patient's pulse. The practitioner may pivot finger guard 134 to an angle that allows the tips of his or her middle and index fingers, extending from finger holes 122, to be placed as close as possible to the desired insertion site for needle 12. However, it should be understood that the practitioner may pivot finger guard 134 to any angle that he or she desires, and may place his or her fingers at any desired distance from the insertion site for needle 12. In any case, finger guard 134 reduces or eliminates the risk of a needlestick injury to the practitioner's fingers by preventing contact between the fingers and needle 12.

The placement of the practitioner's fingers and/or lower component 120 on the patient's skin 20 serves to stabilize platform 132 supporting syringe 10. Thus, the practitioner may stably place device 100 using one hand (e.g., right hand), such that needle 12 of syringe 10 is at the desire angle and on a linear path towards the desired insertion site. Then, the practitioner may use his or her other hand (e.g., left hand) to slide syringe 10 on a downward linear trajectory along the longitudinal axis of platform 132 to thereby push needle 12 into the patient's skin 20 at the desired insertion site. In embodiments with attachment mechanism 600, syringe 10 is restricted to only move along the linear trajectory, thereby reducing or eliminating inadvertent lateral movement of needle 12.

Compared to prior methods of arterial blood sampling (e.g., for ABG determination), use of device 100 decreases the overall time for the procedure. Since ABG determinations are often performed during emergency situations, the decrease in time of the procedure can improve overall patient outcomes. In fact, the improved time can be the difference between a patient living or dying. Furthermore, the decrease in time, for an arterial blood draw performed prior to surgery, advantageously decreases the amount of time that a patient must remain anesthetized.

Currently, a trained healthcare professional—commonly, a physician—is required to perform arterial blood sampling. However, use of device 100 has the potential to enable less experienced practitioners or even trainees to perform arterial blood sampling. Specifically, by increasing accuracy, device 100 can decrease pain and the risk of infection for patients, since fewer punctures translates to fewer infection-prone breaks in the patients' skin 20. Thus, during an operation or emergency, a less skilled healthcare practitioner could perform the arterial blood sampling, while the more experienced healthcare practitioner is freed to use his or her time and expertise to address other issues.

Advantageously, device 100 enables a practitioner to palpate the radial artery pulse, distal to the needle insertion site during insertion of needle 12, thereby eliminating the problem of decreased blood flow to the needle insertion site. Due to the presence of protective finger guard 134 below platform 132 of device 100, the practitioner can insert needle 12 closer to the site of palpation without having to worry about a needlestick injury to the practitioner's fingers. The closer that needle 12 is to the identified pulsation, the greater the chance of successfully puncturing the artery.

Any patient admitted to a hospital or undergoing surgery may benefit from device 100. For example, for surgical patients, the decreased time needed for arterial blood sampling or the insertion of an arterial line decreases the time spent under general anesthesia. Patients under general anesthesia must be intubated. Since intubation raises the risk of numerous complications, decreasing the time a patient spends under general anesthesia—and thus, intubated—can decrease overall morbidity and mortality associated with surgery. For non-surgical patients, quicker, more efficient, and more accurate ABG sampling can decrease both the pain that the patients experience and the patients' risk of injury from exposure to the procedure.

The above description of the disclosed embodiments is provided to enable any person skilled in the art to make or use the invention. Various modifications to these embodiments will be readily apparent to those skilled in the art, and the general principles described herein can be applied to other embodiments without departing from the spirit or scope of the invention. Thus, it is to be understood that the description and drawings presented herein represent a presently preferred embodiment of the invention and are therefore representative of the subject matter which is broadly contemplated by the present invention. It is further understood that the scope of the present invention fully encompasses other embodiments that may become obvious to those skilled in the art and that the scope of the present invention is accordingly not limited.

Combinations, described herein, such as “at least one of A, B, or C,” “one or more of A, B, or C,” “at least one of A, B, and C,” “one or more of A, B, and C,” and “A, B, C, or any combination thereof” include any combination of A, B, and/or C, and may include multiples of A, multiples of B, or multiples of C. Specifically, combinations such as “at least one of A, B, or C,” “one or more of A, B, or C,” “at least one of A, B, and C,” “one or more of A, B, and C,” and “A, B, C, or any combination thereof” may be A only, B only, C only, A and B, A and C, B and C, or A and B and C, and any such combination may contain one or more members of its constituents A, B, and/or C. For example, a combination of A and B may comprise one A and multiple B's, multiple A's and one B, or multiple A's and multiple B's.

1. A device to assist in arterial punctures, the device comprising:
an upper component comprising a syringe platform,
wherein the syringe platform includes a proximal end and a distal end defining a longitudinal axis,
wherein the upper component comprises a finger guard movably attached to the distal end of the syringe platform;
a lower component comprising one or more finger holes; and
a coupling component that couples the lower component to the upper component.

2. The device of claim 1, wherein the distal end is closer to a needle of a syringe when the syringe is supported by the syringe platform.

3. (canceled)

4. The device of claim 3, wherein the finger guard extends parallel to the syringe platform.

5. (canceled)

6. The device of claim 1, wherein the finger guard is capable of pivoting between a range of angles relative to the syringe platform, and wherein the range of angles comprises a 0-degree angle, in which the finger guard is parallel to the syringe platform, and a 90-degree angle, in which the finger guard is perpendicular to the syringe platform.

7. The device of claim 2, wherein the syringe platform comprises an attachment component configured to releasably attach to a corresponding attachment component on the syringe and restrict movement of the syringe to a linear axis that is parallel to the longitudinal axis.

8. The device of claim 7, wherein the attachment component comprises a T-shaped track extending from the proximal end to the distal end of the syringe platform.

9. The device of claim 2, wherein a cross section of the syringe platform, in a plane that is perpendicular to the longitudinal axis, is semi-circular.

10. The device of claim 2, wherein the syringe platform is tapered, such that a cross section of the syringe platform in a plane that is perpendicular to the longitudinal axis consists of a smaller portion of a circle at the proximal end than at the distal end.

11. The device of claim 2, wherein a cross section of the syringe platform, in a plane that is perpendicular to the longitudinal axis, is circular from the proximal end to the distal end, and wherein a diameter of the cross section at the proximal end is not equal to a diameter of the cross section at the distal end.

12. The device of claim 1, wherein the lower component comprises at least two finger holes.

13. The device of claim 12, wherein the lower component consists of two finger holes.

14. The device of claim 12, wherein a first one of the at least two finger holes has a different inner diameter than a second one of the at least two finger holes.

15. The device of claim 14, wherein the first finger hole is configured to receive a human middle finger, and wherein the second finger hole is configured to receive a human index finger.

16. The device of claim 12, wherein each of the at least two finger holes is configured to only surround a proximal phalanx of a human finger.

17. The device of claim 12, wherein each of the at least two finger holes has a proximal end and a distal end, wherein the distal end is closer to a needle of a syringe when the syringe is supported by the syringe platform, and wherein the distal end of each of the at least two finger holes is tapered.

18. The device of claim 17, wherein the distal end of each of the at least two finger holes is tapered, such that a length of each finger hole decreases from a top to a bottom of the finger hole.

19. The device of claim 12, wherein each of the at least two finger holes comprises an annular grip within the finger hole, wherein each annular grip comprises a tube of compressible material configured to receive a human finger therethrough.

20. The device of claim 1, wherein the coupling component is configured to enable movement of the upper component relative to the lower component, when manual force is applied, such that the upper component is movable through a range of angles relative to the lower component, and wherein the coupling component is configured to prevent movement of the upper component relative to the lower component when no manual force is applied.

21. The device of claim 1, wherein the coupling component comprises a ball and socket, such that the upper component is movable through a range of angles in three dimensions relative to the lower component.

22. The device of claim 1, wherein the lower component consists of a single finger hole configured to receive a human finger therethrough.

23. A device to assist in arterial punctures, the device comprising:
an upper component comprising a platform configured to support a syringe,
wherein the platform has a proximal end and a distal end defining a longitudinal axis,
wherein the distal end is closer to a needle of the syringe when the syringe is supported by the platform, and
wherein the upper component further comprises a finger guard that extends from the distal end of the platform, the finger guard being movably attached to the distal end of the platform;
a lower component comprising one or more finger holes,
wherein each of the one or more finger holes is configured to receive a human finger therethrough, so as to enable contemporaneous stabilization of the lower component on the human finger and palpation of an arterial pulse by the human finger during an arterial puncture; and
a coupling component that couples the lower component to the upper component, and
wherein the coupling component comprises a ball and socket, such that the upper component is movable through a range of angles in three dimensions relative to the lower component.

24. The device of claim 23, wherein the finger guard is movably attached to the distal end of the platform, such that the finger guard is capable of pivoting between a range of angles relative to the platform.

25. The device of claim 24, wherein the range of angles comprises a 0-degree angle, in which the finger guard is parallel to the platform, and a 90-degree angle, in which the finger guard is perpendicular to the platform.
